(19) United States
(12) Patent Application Publication
(10) Pub. No.: US 2023/0220211 A1
(43) Pub. Date: Jul. 13, 2023

(54) PICKERING EMULSIONS

(71) Applicant: Omya International AG, Oftringen (CH)

(72) Inventors: Marcel LEX, Hirschau (DE); Florentine HILTY-VANCURA, Meilen (CH); Laura DE MIGUEL, Zofingen (CH); Sylvain MASSE, Morges (CH)

(73) Assignee: Omya International AG, Oftringen (CH)

(21) Appl. No.: 18/000,875

(22) PCT Filed: Jul. 22, 2021
(86) PCT No.: PCT/EP2021/070558

§ 371 Date: Dec. 6, 2022

(30) Foreign Application Priority Data
Jul. 22, 2020 (EP) 20187237.1

(51) Int. Cl. C09C 1/02

(52) U.S. Cl. CPC C09C 1/021; C01P 2004/61; C01P 2006/12; C01P 2006/14

(57) ABSTRACT

The present invention refers to Pickering emulsion comprising (i) water; (ii) 10 to 50 wt.-% oil, based on the total weight of the Pickering emulsion and (iii) 1 to 10 wt.-% of Pickering pigments, based on the total weight of the Pickering emulsion, wherein the Pickering pigments are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from 0.2 µm to 10 µm. Furthermore, the present invention refers to a composition comprising said Pickering emulsions and a method of preparing such Pickering emulsions. The present invention also refers to the use of calcium carbonate particles as Pickering pigments for stabilizing Pickering emulsions comprising water and 10 to 50 wt.-% oil, based on the total weight of the Pickering emulsion, wherein the calcium carbonate particles are selected from surface-reacted calcium carbonate (SRCC) or mixtures of ground calcium carbonate (GCC) and surface-reacted calcium carbonate (SRCC) and have a volume median particle size d₅₀ value from 0.2 µm to 10 µm.

The present invention refers to Pickering emulsion comprising (i) water; (ii) 10 to 50 wt.-% oil, based on the total weight of the Pickering emulsion and (iii) 1 to 10 wt.-% of Pickering pigments, based on the total weight of the Pickering emulsion, wherein the Pickering pigments are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from 0.2 µm to 10 µm. Furthermore, the present invention refers to a composition comprising said Pickering emulsions and a method of preparing such Pickering emulsions. The present invention also refers to the use of calcium carbonate particles as Pickering pigments for stabilizing Pickering emulsions comprising water and 10 to 50 wt.-% oil, based on the total weight of the Pickering emulsion, wherein the calcium carbonate particles are selected from surface-reacted calcium carbonate (SRCC) or mixtures of ground calcium carbonate (GCC) and surface-reacted calcium carbonate (SRCC) and have a volume median particle size d₅₀ value from 0.2 µm to 10 µm.

The term “emulsion” generally relates to heterogonous systems consisting of two immiscible or barely miscible liquids that are typically designated as phases. One of both liquids is dispersed in the other liquid in the form of fine droplets. However, in order to obtain a durable dispersion of a liquid in another liquid, the addition of a surface active agent (emulsifier) is normally required. Such emulsifiers normally have an amphiphile molecular structure consisting of a polar (hydrophile) and a non-polar (lipophile) part of the molecule which are separated from each other in space.

In simple emulsions, finely disperse droplets of one phase, surrounded by an emulsifier shell (water droplets in water-in-oil [W/O] emulsions or lipid vesicles in oil-in-water [O/W] emulsions) are present in the second phase. Emulsifiers lower the interfacial tension between the phases by positioning themselves at the interface between the two liquids. At the phase boundary, they form oil/water interfacial films, which prevent irreversible coalescence of the droplets. Emulsions are frequently stabilized using emulsifier mixtures.

However, such emulsifiers may have disadvantages. Some of the known emulsifiers may be harmful or even toxic to humans or nature. Some emulsifiers may trigger allergies. Furthermore, emulsifiers create additional cost in the end products.

Therefore, emulsifier free emulsions are often used. Such emulsions are a special form of an emulsion. These emulsions are free of emulsifiers in a narrower sense, i.e. free of amphiphilic compounds having a low molecular weight (molecular weight of < 5000) that in higher concentrations form micelles and/or other liquid crystalline aggregates. In addition, these substances may increase the stability of emulsions in that they reduce the rate of aggregation and/or coalescence.

However, another form of emulsions are Pickering emulsions. In the early 1900s, Pickering prepared paraffin/water emulsions that were stabilized merely by the addition of colloidal solids. This type of emulsion is thus also referred to as a Pickering emulsion. In Pickering emulsions, the solids accumulate in the oil/water boundary surface in the form of a layer whereby the joining of the dispersed phases is prevented. In this respect, the wetting properties of the solid particles, which should be wettable by both the hydrophilic as well as the lipophilic phases, are of special importance. Pickering emulsions are encountered in various natural and industrial processes such as crude oil recovery, oil separation, cosmetic preparation, waste water treatment, food compositions etc.

A known Pickering pigment is calcium carbonate. An advantage of calcium carbonate is that it is non-toxic and, therefore, may be used also in cosmetic formulations, in food formulations or compositions that are used in the environment. Such compositions are, for example known from the article “Emulsion phase inversion from oil-in-water (1) to water-in-oil to oil in water (2) induced by in situ surface activation of CaCO₃ nanoparticles via adsorption of sodium stearate” by Zhang et al., Physicochem. Eng. Aspects 477, 2015, pp 55 - 62 that refers to calcium carbonate nanoparticles that are activated as emulsifiers by interaction with sodium stearate and the calcium carbonate nanoparticles have sizes generally between 10 and 100 nm. Another article is “Effect of trace impurities in triglyceride oils on phase inversion of Pickering emulsions stabilized by CaCO₃ nanoparticles” by Zhu et al., Physicochem. Eng. Aspects 417, 2013, pp 126 - 132 that refers to oil in water or water in oil emulsions stabilized by CaCO₃ nanoparticles, wherein the emulsions comprise anionic surfactants such as sodium dodecyl sulfate or sodium carboxylates as hydrophobizing agents in trace amounts that hydrophobize the surface of the CaCO₃ nanoparticles, wherein the nanoparticles have a primary diameter between 80 nm and 120 nm. From the article “Multiple Phase Inversion of Emulsion Stabilized by in Situ surface Activation of CaCO₃ Nanoparticles via Adsorption of Fatty Acids” by Cui et al., Langmuir, 2012, 28, pp 314 - 320 it is known that hydrophilic CaCO₃ nanoparticles that have been surface activated with sodium carboxylates of chain length between 6 and 12 as well as sodium 2-ethylhexylsulfosuccinate can be used to stabilize oil in water or water in oil emulsions at the interface, wherein the calcium carbonate particles are nanoparticles and have a primary diameter between 80 and 120 nm. However, all these Pickering emulsions comprise nanoparticles that are smaller than 120 nm. Recent research has shown that such small nanoparticles may have disadvantages for the environment, for humans and animals. More precisely, these nanoparticles can enter organisms during ingestion or through the skin and can translocate within the body to various organs and tissues or within plants. Due to their reactivity within humans, animals or plants cells they can show toxicological effects. More precisely, such nanoparticles have the ability to organize around proteins and due to this binding, some particles generate adverse biological outcomes through protein unfolding, fibrillation, thiol crosslinking, and loss of enzymatic activity. Therefore, Pickering particles are needed that have primary diameters above 150 nm.

US20100272765 A1 refers to a stable emulsion and process for preparing the same. The solid particulate material in the emulsions has an average particle size of at most 200 nm. The emulsion comprise (a) an oil; (b) water; (c) a surfactant; and (d) solid particulate material, wherein the surfactant can be any anionic, zwitterionic or amphoteric, nonionic or cationic surfactant known to the skilled person. WO2009112836 refers to Pickering emulsion formulations that comprise (a) an aqueous continuous phase; (b) a dispersed oil phase which comprises at least one substantially water-insoluble pesticidally active ingredient; (c) at least one colloidal solid stabilizer, situated at the interface between the continuous and dispersed phases that may have a number-weighted median particle size of 0.5 µm or less; and (d) at least one polymeric co-stabilizer. JP2017508441 A refers to an edible emulsion comprising at least one aqueous phase and at least one lipid phase, the emulsion being stabilized by particles of edible inorganic salt, wherein the edible inorganic salt can be calcium carbonate and the particles have 0.5 to 20% by weight fatty acid coated or adsorbed on the surface thereof. The article “On the Pickering emulsions stabilized by calcium carbonate particles with various morphologies” by Huang Funing et al., Colloids and Surfaces A 580 (2019) 123722, refers to cubic, spherical and rod-like calcium carbonate particles (PCC particles) that are prepared by a precipitation method and are used as stabilizers for the formation of Pickering emulsions. The Pickering emulsions stabilized by calcium carbonate particles are of oil-in-water type. The article “Pickering Emulsions stabilized by Calcium Carbonate Particles: A New Topical Formulation”, Joana Marto et al., Cosmetics 2020, 7, 62, 7030062 refers to calcium carbonate particles as stabilizers of Pickering emulsions for topical use. The formulations prepared in this article have a pH compatible with human skin and a shear thinning behavior and comprise caprylic/capric acid triglyceride and calcium carbonate which is derived from crushing aggregates of limestone and, therefore, is a ground natural calcium carbonate (GCC). The solid particles or pigments that are used as Pickering pigments are often ground calcium carbonate particles or precipitated calcium carbonate particles. However, when PCC or GCC particles are used as Pickering pigments the droplet size is often not very homogeneous and, furthermore, these emulsions are often instable, for example, a separated oil phase can be detected after several days. Therefore, in order to produce stable Pickering emulsions often the addition of surfactants, co-stabilizers or surface coatings on the surface of the Pickering pigments is necessary. The addition of such surfactants, co-stabilizers or surface coatings often is not desirable especially in Pickering emulsions that are used in agriculture or for humans and animals since such compounds can have side effect for humans or animals and may not be environmentally friendly.

Therefore, there is a continuous need in the art for alternative or improved Pickering emulsion.

One object of the present invention may be seen in the provision of a Pickering emulsion that comprises novel Pickering pigments that have not been used as Pickering pigments before. Another object of the present invention may be seen in the provision of Pickering emulsions that do not comprise nanoparticles that have primary diameters below 150 nm. A further object of the present invention may be seen in the provision of a Pickering emulsion wherein the emulsion does not comprise an additional emulsifier for stabilizing the droplets in the Pickering emulsions apart from the Pickering pigments. Another object of the present invention may be seen in the provision of a more environmentally compatible Pickering emulsion. A further object of the present invention may be seen in the provision of Pickering emulsions that can be easily and quickly produced, are cheap and especially easy to handle.

The foregoing and other problems may be solved by the subject-matter as defined herein in the independent claims.

According to one aspect of the present invention, Pickering emulsion are provided, comprising (i) water; (ii) 10 to 50 wt.-% oil, based on the total weight of the Pickering emulsion and (iii) 1 to 10 wt.-% of Pickering pigments, based on the total weight of the Pickering emulsion, wherein the Pickering pigments are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from 0.2 µm to 10 µm.

The inventors of the present invention surprisingly found out that the use of Pickering pigments that are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from 0.2 µm to 10 µm is advantageous for preparing Pickering emulsion. First of all, the Pickering pigments of the present invention have a volume median particle size d₅₀ value from 0.2 µm to 10 µm and, therefore, do not comprise nanoparticles that have mainly primary diameters below 150 nm. Furthermore, the Pickering emulsion of the present invention do not comprise an additional emulsifier for stabilizing the droplets in the Pickering emulsions apart from the Pickering pigments and, therefore, clean label emulsions may be produced. Additionally, the Pickering pigments of the present invention are not toxic or harmful to environment, humans or animals. Furthermore, the inventors found that the inventive Pickering emulsions have a white colour, even if a coloured, for example, a yellow oil is used. Finally, the inventors surprisingly found that the inventive Pickering emulsion can be easily and quickly produced, are cheap and especially easy to handle.

According to another aspect of the present invention, a composition is provided comprising the inventive Pickering emulsion, wherein the composition is a food composition, a cosmetic composition, a pharmaceutical composition or a nutritional formula.

According to another aspect of the present invention, a method of preparing a Pickering emulsion is provided, the method comprising the steps of: A) providing water, B) providing oil, C) providing Pickering pigments, wherein the Pickering pigments are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from above 0.1 µm to 10 µm, D) combining the water of step A), the oil of step B) and the Pickering pigments of step C) in any order to obtain a mixture comprising 10 to 50 wt.-% oil, based on the total weight of the mixture and 1 to 10 wt.-% of Pickering pigments, based on the total weight of the mixture and E) mixing the mixture obtained in step D) to prepare a Pickering emulsion.

According to another aspect of the present invention, calcium carbonate particles are used as Pickering pigments for stabilizing Pickering emulsions comprising water and 10 to 50 wt.-% oil, based on the total weight of the Pickering emulsion, wherein the calcium carbonate particles are selected from surface-reacted calcium carbonate (SRCC) or mixtures of ground calcium carbonate (GCC) and surface-reacted calcium carbonate (SRCC) and have a volume median particle size d₅₀ value from 0.2 µm to 10 µm.

Advantageous embodiments of the above aspects are defined in the corresponding subclaims.

According to one embodiment of the present invention, the ground calcium carbonate is selected from the group consisting of marble, limestone, and/or chalk and preferably is marble and/or
the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

According to another embodiment of the present invention, the ground calcium carbonate has
a) a volume median particle size d₅₀ value from 0.3 µm to 5.0 µm, preferably from 0.6 µm to 3 µm and most preferably from above 1.0 µm to 1.7 µm, and/or
b) a top cut (d₉₈(vol)) of ≤ 20 µm, preferably ≤ 15 µm, more preferably ≤ 10 µm and most preferably ≤ 7 µm, and/or
c) a specific surface area (BET) of from 0.5 to 50 m²/g, preferably from 0.5 to 35 m²/g, more preferably from 0.5 to 25 m²/g, and most preferably from 0.6 to 17 m²/g, as measured by the BET nitrogen method.

According to another embodiment of the present invention, the surface-reacted calcium carbonate has
a) a volume median particle size d₅₀ value from 1.5 µm to 9.0 µm, preferably from 2.5 µm to 7.5 µm and most preferably from 3.3 µm to 6.6 µm, and/or
b) a top cut (d₉₈(vol)) of ≤ 20 µm, preferably ≤ 15 µm, more preferably ≤ 10 µm and most preferably ≤ 7 µm, and/or
c) a specific surface area (BET) of from 10 to 200 m²/g, preferably from 20 to 180 m²/g, more preferably from 25 to 140 m²/g, and most preferably from 48 to 110 m²/g, as measured by the BET nitrogen method and/or
d) an intra-particle intruded specific pore volume in the range from 0.1 to 2.3 cm³/g, more preferably from 0.2 to 2.0 cm³/g, especially preferably from 0.4 to 1.5 cm³/g, and most preferably from 0.6 to 1.1 cm³/g, calculated from mercury porosimetry measurement.

According to another embodiment of the present invention, the emulsions comprise 10 to 40 wt.-% oil, based on the total weight of the Pickering emulsion, preferably 10 to 30 wt.-% oil and most preferably 10 to 20 wt.-% oil.

According to another embodiment of the present invention, the oil
is selected from the group consisting of mineral oils, vegetable oils, animal fats, essential oils and mixtures thereof, preferably selected from the group consisting of essential oils, sunflower oil, olive oil, palm oil, coconut oil, peanut oil, palm kernel oil, corn oil, hazelnut oil, sesame oil and mixtures thereof, preferably is selected from sunflower oil, olive oil, palm oil and/or coconut oil and most preferably is sunflower oil and/or
is a refined oil having an acid value below 0.6, preferably below 0.5 and most preferably below 0.3 or an unrefined oil having an acid value below 4.0, preferably below 3.0 and most preferably below 2.0.

According to another embodiment of the present invention, the emulsions comprise 2 to 10 wt.-% Pickering pigments, based on the total weight of the Pickering emulsion, preferably 4 to 10 wt.-% Pickering pigments and most preferably 6 to 10 wt.-% Pickering pigments.

According to another embodiment of the present invention, the emulsions comprise further active ingredients, preferably selected from cosmetic active compounds, pharmaceutical active compounds, nutritional additives, flavoring agents and mixtures thereof.

According to another embodiment of the present invention, the emulsions are stable against coalescence for at least 15 days, more preferably for at least 20 days and most preferably for at least 30 days.

According to another embodiment of the present invention, the emulsion does not comprise an additional emulsifier for stabilizing the droplets in the Pickering emulsions apart from the Pickering pigments.

According to another embodiment of the present invention, the surface of the Pickering pigments provided in step C) and preferably of the surface-reacted calcium carbonate particles is not coated with a surface treatment agent.

An “emulsion” in the meaning of the present invention refers to a mixture of two or more liquids that are normally immiscible and wherein one liquid is dispersed in the other liquid. A “Pickering emulsion” in the meaning of the present invention is an emulsion wherein the Pickering pigments accumulate in the oil/water boundary surface in the form of a layer whereby the joining of the dispersed phases is prevented. A “Pickering pigment” in the meaning of the present invention is a pigment that accumulates in the oil/water boundary surface of the droplets in the Pickering emulsion and stabilizes them. A “pigment” in the meaning of the present invention is an inorganic solid material having a defined chemical composition and a characteristic crystalline structure. Pigments are insoluble in water and oil.

An oil in the meaning of the present invention is a compound that is liquid at 25° C. and 1.0 bar and does not form a homogeneous mixture when mixed with water.

“Ground natural calcium carbonate” (GNCC) in the meaning of the present invention is a calcium carbonate obtained from natural sources, such as limestone, marble, or chalk, and processed through a wet and/or dry treatment such as grinding, screening and/or fractionation, for example, by a cyclone or classifier.

“Precipitated calcium carbonate” (PCC) in the meaning of the present invention is a synthesised material, generally obtained by precipitation following a reaction of carbon dioxide and calcium hydroxide (hydrated lime) in an aqueous environment or by precipitation of a calcium- and a carbonate source in water. Additionally, precipitated calcium carbonate can also be the product of introducing calcium- and carbonate salts, calcium chloride and sodium carbonate for example, in an aqueous environment. PCC may have a vateritic, calcitic or aragonitic crystalline form. PCCs are described, for example, in EP2447213 A1, EP2524898 A1, EP2371766 A1, EP2840065 A1, or WO2013142473 A1.

A “surface-reacted calcium carbonate” according to the present invention is a reaction product of ground natural calcium carbonate (GNCC) or precipitated calcium carbonate (PCC) treated with CO₂ and one or more H₃O⁺ ion donors, wherein the CO₂ is formed in situ by the H₃O⁺ ion donors treatment and/or is supplied from an external source. A H₃O⁺ ion donor in the context of the present invention is a Bronsted acid and/or an acid salt.

The “particle size” of the Pickering pigments is described as volume-based particle size distribution dₓ(vol). Therein, the value dx(vol) represents the diameter relative to which x% by volume of the particles have diameters less than dₓ(vol). This means that, for example, the d₂₀(vol) value is the particle size at which 20 vol.-% of all particles are smaller than that particle size. The d₅₀(vol) value is thus the volume median particle size, i.e. 50 vol.-% of all particles are smaller than that particle size and the d₉₈(vol) value is the particle size at which 98 vol.-% of all particles are smaller than that particle size. The volume median particle size d₅₀ was evaluated using a Malvern Mastersizer 2000 Laser Diffraction System. The raw data obtained by the measurement are analysed using the Mie theory, with a particle refractive index of 1.57 and an absorption index of 0.005.

The “particle size” of other materials than the Pickering pigments, for example the ground calcium carbonate or the precipitated calcium carbonate that is used for preparing the surface-reacted calcium carbonate, is described by its distribution of particle sizes dₓ(wt). Therein, the value dx(wt) represents the diameter relative to which x% by weight of the particles have diameters less than dₓ(wt). This means that, for example, the d₂₀(wt) value is the particle size at which 20 wt.-% of all particles are smaller than that particle size. The d₅₀(wt) value is thus the weight median particle size, i.e. 50 wt.-% of all particles are smaller than that particle size. The measurement is made with a Sedigraph™ 5120 of Micromeritics Instrument Corporation, USA. The method and the instrument are known to the skilled person and are commonly used to determine particle size distributions. The measurement is carried out in an aqueous solution of 0.1 wt.% Na₄P₂O₇. The samples are dispersed using a high speed stirrer and sonication.

Throughout the present document, the “specific surface area” (in m²/g) of the Pickering pigments or other materials is determined using the BET method (using nitrogen as adsorbing gas), which is well known to the skilled man (ISO 9277:2010).

For the purpose of the present invention the “porosity” or “pore volume” refers to the intra-particle intruded specific pore volume. Said porosity or pore volume is measured using a Micromeritics Autopore V 9620 mercury porosimeter.

“Coalescence” in the meaning of the present invention refers to the disappearance of the boundary between two droplets in contact to form a single droplet, followed by changes of shape leading to a reduction of the total surface area.

A “suspension” or “slurry” in the meaning of the present invention comprises insoluble solids and a liquid medium, for example water, and optionally further additives, and usually contains large amounts of solids and, thus, is more viscous and can be of higher density than the liquid from which it is formed.

The term “solid” according to the present invention refers to a material that is solid under standard ambient temperature and pressure (SATP) which refers to a temperature of 298.15 K (25° C.) and an absolute pressure of exactly 1 bar. The solid may be in the form of a powder, tablet, granules, flakes etc. Accordingly, the term “liquid medium” refers to a material that is liquid under standard ambient temperature and pressure (SATP) which refers to a temperature of 298.15 K (25° C.) and an absolute pressure of exactly 1 bar.

Where the term “comprising” is used in the present description and claims, it does not exclude other non-specified elements of major or minor functional importance. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Whenever the terms “including” or “having” are used, these terms are meant to be equivalent to “comprising” as defined above.

Where an indefinite or definite article is used when referring to a singular noun, e.g. “a”, “an” or “the”, this includes a plural of that noun unless something else is specifically stated.

Terms like “obtainable” or “definable” and “obtained” or “defined” are used interchangeably. This, e.g., means that, unless the context clearly dictates otherwise, the term “obtained” does not mean to indicate that, e.g., an embodiment must be obtained by, e.g. the sequence of steps following the term “obtained” even though such a limited understanding is always included by the terms “obtained” or “defined” as a preferred embodiment.

As set out hereinabove, the present invention refers to a Pickering emulsion comprising
(i) water;
(ii) 10 to 50 wt.-% oil, based on the total weight of the Pickering emulsion and
(iii) 1 to 10 wt.-% of Pickering pigments, based on the total weight of the Pickering emulsion,
   wherein the Pickering pigments are calcium carbonate particles selected from surface-reacted calcium carbonate (SRCC) or mixtures of ground calcium carbonate (GCC) and surface-reacted calcium carbonate (SRCC) and
   wherein the calcium carbonate particles have a volume median particle size d₅₀ value from 0.2 µm to 10 µm.

In the following, details and preferred embodiments of the Pickering emulsions will be set out in more detail. It is to be understood that these embodiments or details apply also for the inventive compositions, the method for preparing the Pickering emulsions and the use of the inventive calcium carbonate particles.

### (I) Water

According to the present invention water is present in the Pickering emulsion.

The water of the present invention may be selected from drinking water, process water, demineralized water, distilled water, rain water, recycled water, river water and mixtures thereof. According to a preferred embodiment of the present invention the water present in the Pickering emulsions is drinking water, demineralized water or distilled water and preferably demineralized water.

Drinking water, also known as potable water, is water that is safe to drink or to use for food preparations. Rain water/river water is obtained from rain/rivers. Recycled water is water that has been recycled and can be used in agriculture. Process water is water which is not considered drinkable and is basically used in relation to industrial plants, industrial processes and production facilities. Demineralized water is specially purified water that has had most or all of its mineral and salt ions removed, such as calcium, magnesium, sodium, chloride, sulphate, nitrate and bicarbonate. It is also known as deionized water. Distilled water is water that has been boiled into vapor and condensed back into liquid in a separate container.

According to one embodiment of the present invention the water is present in the Pickering emulsion in an amount from 11 to 80 wt.-% based on the total weight of the Pickering emulsion, preferably in an amount of 20 to 80 wt.-%, even more preferably in an amount of 30 to 70 wt.-% and most preferably in an amount of 40 to 60 wt.-% based on the total weight of the Pickering emulsions.

### (II) Oil

According to the present invention, oil is present in the Pickering emulsion.

The oil is liquid at 25° C. and 1.0 bar and does not form a homogeneous mixture when mixed with the water.

The oil is present in the Pickering emulsion in an amount of 10 to 50 wt.-% oil, based on the total weight of the Pickering emulsion. According to a preferred embodiment of the present invention the Pickering emulsions comprise 10 to 40 wt.-% oil, based on the total weight of the Pickering emulsion, preferably 10 to 30 wt.-% oil and most preferably 10 to 20 wt.-% oil.

The ration of oil : water in the Pickering emulsions may be from 100:600 to 100:20, preferably from 100:400 to 100:40, more preferably from 100:200 to 100:60 and most preferably from 100:150 to 100:80, based on the weight of the water and the oil.

In one embodiment of the present invention, the Pickering emulsion comprises only one oil. Alternatively, the Pickering emulsion comprises two or more different oils. For example, the Pickering emulsion comprises two or three different oils. If the Pickering emulsion comprises more than one oil the different oils may be miscible or immiscible but preferably are miscible.

The oil may be any oil known to the skilled person that is suitable for the particular application. Such oils are commercially available.

According to one embodiment of the present invention the oil is selected from the group consisting of mineral oils, vegetable oils, animal fats, essential oils and mixtures thereof.

“Mineral oils” in the meaning of the present invention are various colorless, odorless, light mixtures of higher alkanes and/or cycloalkanes from a mineral source, particularly a distillate of petroleum. It has a density of around 0.8-0.87 g/cm³. Mineral oils are also known as white oil, paraffin oil, liquid paraffin, paraffinum liquidum, and liquid petroleum. Mineral oil is a liquid by-product of refining crude oil to make gasoline and other petroleum products. Mineral oils are known to the skilled person and are commercially available.

“Vegetable oils” also known as vegetable fats in the meaning of the present invention are oils extracted from seeds or from other parts of fruits. Vegetable fats are mostly a mixture of triglycerides. Vegetable oils are usually edible. Vegetable oils are known to the skilled person and are commercially available. Common vegetable oils are, for example, sunflower oil, olive oil, palm oil, coconut oil, peanut oil, palm kernel oil, corn oil, hazelnut oil, sesame oil, avocado oil, babassu oil, rice bran oil or castor oil.

“Animal fats” also known as animal oils in the meaning of the present invention are lipid materials derived from animals and are often composed of triglycerides. Although many animal parts and secretions may yield oil, in commercial practice, oil is extracted primarily from rendered tissue fats obtained from livestock animals. However, also dairy products yield popular animal fat and oil products such as cheese, butter, and milk. Animal oils are known to the skilled person and are commercially available. Common animals oils are, for example, fish oil, lard oil, mink oil or cod liver oil.

“Essential oils” also known as volatile oils, ethereal oils or aetherolea in the meaning of the present invention are concentrated hydrophobic liquid containing volatile chemical compounds from plants. Essential oils contain the “essence of” the plant’s fragrance—the characteristic fragrance of the plant from which it is derived. Essential oils are generally extracted by distillation, often by using steam. Essential oils are known to the skilled person and are commercially available. Common essential oils are, for example, sweet orange oil, peppermint oil, rose oil, neem oil, lavender oil, lemon oil, rosemary oil, pine oil, tea tree oil, clove oil or jasmine oil.

According to a preferred embodiment of the present invention the oils are selected from the group consisting of essential oils, sunflower oil, olive oil, palm oil, coconut oil, peanut oil, palm kernel oil, corn oil, hazelnut oil, sesame oil and mixtures thereof, preferably is selected from sunflower oil, olive oil, palm oil and/or coconut oil and most preferably is sunflower oil.

Additionally or alternatively, the oil is a refined oil. A refined oil in the meaning of the present invention is an oil that has been obtained from a “cleaning” process that may involve de-gumming, neutralizing, bleaching and/or deodorizing these oils. Such cleaning processes are known to the skilled person and depend on the oil and the subsequent application and are known to the skilled person. The refined oils have an acid value below 0.6, preferably below 0.5 and most preferably below 0.3.

However, the oil may also be an unrefined oil having an acid value below 4.0, preferably below 3.0 and most preferably below 2.0.

The “acid value” also known as “neutralization number”, “acid number” or “acidity” is the mass of potassium hydroxide (KOH) in milligrams that is required to neutralize one gram of chemical substance, e.g. the oil. The acid number is a measure of the number of carboxylic acid groups in a chemical compound, such as the oil. The skilled person known how to measure the acid value.

According to one embodiment of the present invention, the oil is selected from the group consisting of mineral oils, vegetable oils, animal fats, essential oils and mixtures thereof, preferably selected from the group consisting of essential oils, sunflower oil, olive oil, palm oil, coconut oil, peanut oil, palm kernel oil, corn oil, hazelnut oil, sesame oil and mixtures thereof, preferably is selected from sunflower oil, olive oil, palm oil and/or coconut oil and most preferably is sunflower oil and is a refined oil having an acid value below 0.6, preferably below 0.5 and most preferably below 0.3 or an unrefined oil having an acid value below 4.0, preferably below 3.0 and most preferably below 2.0.

According to an exemplified embodiment of the present invention the oil is a vegetable oil and preferably sunflower oil. Sunflower oil is commercially available, for example from M-classic.

### (III) Pickering Pigments

According to the present invention, the Pickering emulsion comprises Pickering pigments.

As already set out above a “Pickering pigment” in the meaning of the present invention is a pigment that accumulates in the oil/water boundary surface of the droplets in the Pickering emulsion and stabilizes them.

The Pickering pigments of the present invention are calcium carbonate particles selected from surface-reacted calcium carbonate (SRCC) or mixtures of ground calcium carbonate (GCC) and surface-reacted calcium carbonate (SRCC), wherein the calcium carbonate particles have a volume median particle size d₅₀ value from 0.2 µm to 10 µm.

According to one embodiment of the present invention, the ground calcium carbonate in the Pickering emulsions is selected from the group consisting of marble, limestone, and/or chalk and preferably is marble; and/or the surface-reacted calcium carbonate in the Pickering emulsions is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

### The Surface-Reacted Calcium Carbonate

A H₃O⁺ ion donor in the context of the present invention is a Bronsted acid and/or an acid salt.

In a preferred embodiment of the invention the surface-reacted calcium carbonate is obtained by a process comprising the steps of: (a) providing a suspension of natural or precipitated calcium carbonate, (b) adding at least one acid having a pKₐ value of 0 or less at 20° C. or having a pKₐ value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO₂, (D) contacting said natural or precipitated calcium carbonate of step (A) with the at least one acid of step (B) and with the CO₂ of step (C), characterised in that: (i) the at least one acid of step B) has a pKₐ of greater than 2.5 and less than or equal to 7 at 20° C., associated with the ionisation of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKₐ of greater than 7 at 20° C., associated with the ionisation of the first available hydrogen, and the salt anion of which is capable of forming water-insoluble calcium salts, is additionally provided.

“Natural ground calcium carbonate” (GCC) used for preparing the surface-reacted calcium carbonate preferably is selected from calcium carbonate containing minerals selected from the group comprising marble, chalk, limestone and mixtures thereof. Natural calcium carbonate may comprise further naturally occurring components such as magnesium carbonate, alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) used for preparing the surface-reacted calcium carbonate in the meaning of the present invention is a synthesized material, generally obtained by precipitation following reaction of carbon dioxide and calcium hydroxide in an aqueous environment or by precipitation of calcium and carbonate ions, for example CaCl₂ and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H₃O⁺ ion donor by the same means as used for grinding natural calcium carbonate as described above.

According to one embodiment of the present invention, the natural or precipitated calcium carbonate used for preparing the surface-reacted calcium carbonate is in form of particles having a weight median particle size d₅₀ of 0.05 to 10.0 µm, preferably 0.2 to 5.0 µm, more preferably 0.4 to 3.0 µm, most preferably 0.6 to 1.2 µm, especially 0.7 µm. According to a further embodiment of the present invention, the natural or precipitated calcium carbonate used for preparing the surface-reacted calcium carbonate is in form of particles having a top cut particle size d₉₈(wt) of 0.15 to 55 µm, preferably 1 to 40 µm, more preferably 2 to 25 µm, most preferably 3 to 15 µm, especially 4 µm.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donor used for the preparation of surface reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acidic salt, generating H₃O⁺ ions under the preparation conditions.

According to one embodiment, the at least one H₃O⁺ ion donor is a strong acid having a pKₐ of 0 or less at 20° C.

According to another embodiment, the at least one H₃O⁺ ion donor is a medium-strong acid having a pKₐ value from 0 to 2.5 at 20° C. If the pKₐ at 20° C. is 0 or less, the acid is preferably selected from sulphuric acid, hydrochloric acid, or mixtures thereof. If the pKₐ at 20° C. is from 0 to 2.5, the H₃O⁺ ion donor is preferably selected from H₂SO₃, H₃PO₄, oxalic acid, or mixtures thereof. The at least one H₃O⁺ ion donor can also be an acidic salt, for example, HSO₄⁻ or H₂PO₄⁻, being at least partially neutralized by a corresponding cation such as Li⁺, Na⁺ or K⁺, or HPO₄²⁻, being at least partially neutralised by a corresponding cation such as Li⁺, Na⁺, K⁺, Mg²⁺ or Ca²⁺. The at least one H₃O⁺ ion donor can also be a mixture of one or more acids and one or more acidic salts.

According to still another embodiment, the at least one H₃O⁺ ion donor is a weak acid having a pKₐ value of greater than 2.5 and less than or equal to 7, when measured at 20° C., associated with the ionisation of the first available hydrogen, and having a corresponding anion, which is capable of forming water-soluble calcium salts. Subsequently, at least one water-soluble salt, which in the case of a hydrogen-containing salt has a pKₐ of greater than 7, when measured at 20° C., associated with the ionisation of the first available hydrogen, and the salt anion of which is capable of forming water-insoluble calcium salts, is additionally provided. According to the preferred embodiment, the weak acid has a pKₐ value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H₃O⁺ ion donor is selected from the group consisting of hydrochloric acid, sulphuric acid, sulphurous acid, phosphoric acid, citric acid, oxalic acid, acetic acid, formic acid, and mixtures thereof. Preferably the at least one H₃O⁺ ion donor is selected from the group consisting of hydrochloric acid, sulphuric acid, sulphurous acid, phosphoric acid, oxalic acid, H₂PO₄⁻, being at least partially neutralised by a corresponding cation such as Li⁺, Na⁺ or K⁺, HPO₄²⁻, being at least partially neutralised by a corresponding cation such as Li⁺, Na⁺, K⁺, Mg²⁺, or Ca²⁺ and mixtures thereof, more preferably the at least one acid is selected from the group consisting of hydrochloric acid, sulphuric acid, sulphurous acid, phosphoric acid, oxalic acid, or mixtures thereof, and most preferably, the at least one H₃O⁺ ion donor is phosphoric acid.

The one or more H₃O⁺ ion donor can be added to the suspension as a concentrated solution or a more diluted solution. Preferably, the molar ratio of the H₃O⁺ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H₃O⁺ ion donor to the water before the natural or precipitated calcium carbonate is suspended.

In a next step, the natural or precipitated calcium carbonate is treated with carbon dioxide. If a strong acid such as sulphuric acid or hydrochloric acid is used for the H₃O⁺ ion donor treatment of the natural or precipitated calcium carbonate, the carbon dioxide is automatically formed. Alternatively or additionally, the carbon dioxide can be supplied from an external source.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKₐ in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor treatment step and/or the carbon dioxide treatment step are repeated at least once, more preferably several times. According to one embodiment, the at least one H₃O⁺ ion donor is added over a time period of at least about 5 min, preferably at least about 10 min, typically from about 10 to about 20 min, more preferably about 30 min, even more preferably about 45 min, and sometimes about 1 h or more.

Subsequent to the H₃O⁺ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

Further details about the preparation of the surface-reacted natural calcium carbonate are disclosed in WO0039222 A1, WO2004083316 A1, WO2005121257 A2, WO2009074492 A1, EP2264108 A1, EP2264109 A1 and US20040020410 A1, the content of these references herewith being included in the present application.

Similarly, surface-reacted precipitated calcium carbonate is obtained. As can be taken in detail from WO2009074492 A1, surface-reacted precipitated calcium carbonate is obtained by contacting precipitated calcium carbonate with H₃O⁺ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H₃O⁺ ions, where said H₃O⁺ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H₃O⁺ ions may be provided by the addition of an acid or an acid salt of said anion, or the addition of an acid or an acid salt which simultaneously serves to provide all or part of said excess solubilized calcium ions.

In a further preferred embodiment of the preparation of the surface-reacted natural or precipitated calcium carbonate, the natural or precipitated calcium carbonate is reacted with the one or more H₃O⁺ ion donors and/or the carbon dioxide in the presence of at least one compound selected from the group consisting of silicate, silica, aluminium hydroxide, earth alkali aluminate such as sodium or potassium aluminate, magnesium oxide, or mixtures thereof. Preferably, the at least one silicate is selected from an aluminium silicate, a calcium silicate, or an earth alkali metal silicate. These components can be added to an aqueous suspension comprising the natural or precipitated calcium carbonate before adding the one or more H₃O⁺ ion donors and/or carbon dioxide.

Alternatively, the silicate and/or silica and/or aluminium hydroxide and/or earth alkali aluminate and/or magnesium oxide component(s) can be added to the aqueous suspension of natural or precipitated calcium carbonate while the reaction of natural or precipitated calcium carbonate with the one or more H₃O⁺ ion donors and carbon dioxide has already started. Further details about the preparation of the surface-reacted natural or precipitated calcium carbonate in the presence of at least one silicate and/or silica and/or aluminium hydroxide and/or earth alkali aluminate component(s) are disclosed in WO2004083316 A1, the content of this reference herewith being included in the present application.

In a particularly preferred embodiment of the present invention, the surface-reacted calcium carbonate is a reaction product of natural ground calcium carbonate with carbon dioxide and one or more H₃O⁺ ion donors, wherein the carbon dioxide is formed in situ by the H₃O⁺ ion donors treatment, and wherein the one or more H₃O⁺ ion donor is phosphoric acid.

The surface-reacted calcium carbonate can be kept in suspension, optionally further stabilised by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

In a preferred embodiment, the surface-reacted calcium carbonate has a specific surface area of from 10 m²/g to 200 m²/g, preferably from 20 m²/g to 180 m²/g, more preferably from 25 m²/g to 140 m²/g, and most preferably from 48 m²/g to 110 m²/g, measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m²/g.

It is furthermore preferred that the surface-reacted calcium carbonate particles have a volume median grain diameter d₅₀ (wt) of from 1.5 to 9 µm, preferably from 2.5 to 7.5 µm, and most preferably from 3.3 to 6.6 µm.

It may furthermore be preferred that the surface-reacted calcium carbonate particles have a top cut diameter d₉₈ (vol) of ≤ 20 µm, preferably ≤ 15 µm, more preferably ≤ 10 µm and most preferably ≤ 7 µm.

The value dₓ represents the diameter relative to which x % by volume of the particles have diameters less than dₓ. This means that the d₉₈ value is the particle size at which 98 % by volume of all particles are smaller. The d₉₈ value is also designated as “top cut”. The dₓ values are given in volume percent. The d₅₀ (vol) value is thus the median particle size, i.e. 50 vol.-% of all grains are smaller than this particle size.

The volume median particle size is evaluated using a Malvern Mastersizer 2000 Laser Diffraction System. The raw data obtained by the measurement are analysed using the Mie theory, with a particle refractive index of 1.57 and an absorption index of 0.005.

The processes and instruments are known to the skilled person and are commonly used to determine grain size of fillers and pigments.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 µm (~ nm). The equilibration time used at each pressure step is 20 seconds. The sample material is sealed in a 5 cm³ chamber powder penetrometer for analysis. The data are corrected for mercury compression, penetrometer expansion and sample material compression using the software Pore-Comp (Gane, P.A.C., Kettle, J.P., Matthews, G.P. and Ridgway, C.J., “Void Space Structure of Compressible Polymer Spheres and Consolidated Calcium Carbonate Paper-Coating Formulations”, Industrial and Engineering Chemistry Research, 35(5), 1996, p1753-1764.).

The total pore volume seen in the cumulative intrusion data can be separated into two regions with the intrusion data from 214 µm down to about 1 - 4 µm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Preferably, the surface-reacted calcium carbonate has an intra-particle intruded specific pore volume in the range from 0.1 to 2.3 cm³/g, more preferably from 0.2 to 2.0 cm³/g, especially preferably from 0.4 to 1.5 cm³/g and most preferably from 0.6 to 1.1 cm³/g, calculated from mercury porosimetry measurement.

The intra-particle pore size of the surface-reacted calcium carbonate preferably is in a range of from 0.004 to 1.6 µm, more preferably in a range of from 0.005 to 1.3 µm, especially preferably from 0.006 to 1.15 µm and most preferably of 0.007 to 1.0 µm, determined by mercury porosimetry measurement.

According to one embodiment of the present invention, the surface-reacted calcium carbonate has
a) a volume median particle size d₅₀ value from 1.5 µm to 9.0 µm, preferably from 2.5 µm to 7.5 µm and most preferably from 3.3 µm to 6.6 µm, and
b) a top cut (d₉₈(vol)) of ≤ 20 µm, preferably ≤ 15 µm, more preferably ≤ 10 µm and most preferably ≤ 7 µm, and
c) a specific surface area (BET) of from 10 to 200 m²/g, preferably from 20 to 180 m²/g, more preferably from 25 to 140 m²/g, and most preferably from 48 to 110 m²/g, as measured by the BET nitrogen method and
d) an intra-particle intruded specific pore volume in the range from 0.1 to 2.3 cm³/g, more preferably from 0.2 to 2.0 cm³/g, especially preferably from 0.4 to 1.5 cm³/g, and most preferably from 0.6 to 1.1 cm³/g, calculated from mercury porosimetry measurement.

According to another embodiment of the present invention, the surface-reacted calcium carbonate has
a) a volume median particle size d₅₀ value from 1.5 µm to 9.0 µm, preferably from 2.5 µm to 7.5 µm and most preferably from 3.3 µm to 6.6 µm, or
b) a top cut (d₉₈(vol)) of ≤ 20 µm, preferably ≤ 15 µm, more preferably ≤ 10 µm and most preferably ≤ 7 µm, or
c) a specific surface area (BET) of from 10 to 200 m²/g, preferably from 20 to 180 m²/g, more preferably from 25 to 140 m²/g, and most preferably from 48 to 110 m²/g, as measured by the BET nitrogen method or
d) an intra-particle intruded specific pore volume in the range from 0.1 to 2.3 cm³/g, more preferably from 0.2 to 2.0 cm³/g, especially preferably from 0.4 to 1.5 cm³/g, and most preferably from 0.6 to 1.1 cm³/g, calculated from mercury porosimetry measurement.

According to an exemplified embodiment of the present invention the Pickering pigments of the present invention are surface-reacted calcium carbonate particles that have a specific surface area of from 10 m²/g to 200 m²/g, preferably from 20 m²/g to 180 m²/g, more preferably from 25 m²/g to 140 m²/g, for example, from 40 m²/g to 70 m²/g, measured using nitrogen and the BET method. Additionally, or alternatively, the surface-reacted calcium carbonate particles have a volume median particle size d₅₀ value from 1.5 µm to 9.0 µm, for example, from 5.0 µm to 8.0 µm. According to a preferred embodiment of the present invention, the Pickering pigments are surface-reacted calcium carbonate particles that have a specific surface area of from 40 m²/g to 70 m²/g, measured using nitrogen and the BET method and a volume median particle size d₅₀ value from 5.0 µm to 8.0 µm.

### The Ground Calcium Carbonate

According to a preferred embodiment of the present invention the ground calcium carbonate is selected from the group consisting of marble, limestone, and/or chalk, and preferably is marble.

As already set out above GCC is understood to be a naturally occurring form of calcium carbonate, mined from sedimentary rocks such as limestone or chalk, or from metamorphic marble rocks and processed through a treatment such as grinding, screening and/or fractionizing in wet and/or dry form, for example by a cyclone or classifier.

The ground calcium carbonate is preferably in the form of a particulate material, and preferably has a volume median particle size d₅₀ value from 0.3 µm to 5.0 µm, preferably from 0.6 µm to 3 µm and most preferably from above 1.0 µm to 1.7 µm.

Additionally or alternatively, the ground calcium carbonate has a top cut (d₉₈(vol)) of ≤ 20 µm, preferably ≤ 15 µm, more preferably ≤ 10 µm and most preferably ≤ 7 µm.

Additionally or alternatively the ground calcium carbonate has a BET specific surface area of from 0.5 to 50 m²/g as measured by the BET nitrogen method. For example, the at least one calcium carbonate has a specific surface area (BET) of from 0.5 to 35 m²/g, more preferably of from 0.5 to 25 m²/g and most preferably of from 0.6 to 17 m²/g as measured by the BET nitrogen method.

According to one embodiment of the present invention, the ground calcium carbonate has
a) a volume median particle size d₅₀ value from 0.3 µm to 5.0 µm, preferably from 0.6 µm to 3 µm and most preferably from above 1.0 µm to 1.7 µm, and
b) a top cut (d₉₈(vol)) of ≤ 20 µm, preferably ≤ 15 µm, more preferably ≤ 10 µm and most preferably ≤ 7 µm, and
c) a specific surface area (BET) of from 0.5 to 50 m²/g, preferably from 0.5 to 35 m²/g, more preferably from 0.5 to 25 m²/g, and most preferably from 0.6 to 17 m²/g, as measured by the BET nitrogen method.

According to another embodiment of the present invention, the ground calcium carbonate has
a) a volume median particle size d₅₀ value from 0.3 µm to 5.0 µm, preferably from 0.6 µm to 3 µm and most preferably from above 1.0 µm to 1.7 µm, or
b) a top cut (d₉₈(vol)) of ≤ 20 µm, preferably ≤ 15 µm, more preferably ≤ 10 µm and most preferably ≤ 7 µm, or
c) a specific surface area (BET) of from 0.5 to 50 m²/g, preferably from 0.5 to 35 m²/g, more preferably from 0.5 to 25 m²/g, and most preferably from 0.6 to 17 m²/g, as measured by the BET nitrogen method.

The ground calcium carbonate (GCC) can be added as a dry material or can be added in wet form, for example, in form of a slurry. It is preferred that the ground calcium carbonate is a dry ground material, a material being wet ground and dried or a mixture of the foregoing materials. In general, the grinding step can be carried out with any conventional grinding device, for example, under conditions such that refinement predominantly results from impacts with a secondary body, i.e. in one or more of: a ball mill, a rod mill, a vibrating mill, a roll crusher, a centrifugal impact mill, a vertical bead mill an attrition mill, a pin mill, a hammer mill, a pulveriser, a shredder, a de-clumper, a knife cutter, or other such equipment known to the skilled man.

In case the ground calcium carbonate is a wet ground calcium carbonate, the grinding step may be performed under conditions such that autogenous grinding takes place and/or by horizontal ball milling, and/or other such processes known to the skilled man. The wet processed ground calcium carbonate thus obtained may be washed and dewatered by well-known processes, e.g. by flocculation, filtration or forced evaporation prior to drying. The subsequent step of drying may be carried out in a single step such as spray drying, or in at least two steps, e.g. by applying a first heating step to the calcium carbonate in order to reduce the associated moisture content to a level which is not greater than about 1 wt.-%, based on the total dry weight of the calcium carbonate. The residual total moisture content of the filler can be measured by the Karl Fischer coulometric titration method, desorbing the moisture in an oven at 195° C. and passing it continuously into the KF coulometer (Mettler Toledo coulometric KF Titrator C30, combined with Mettler oven DO 0337) using dry N₂ at 100 ml/min for 10 min. The residual total moisture content can be determined with a calibration curve and also a blind of 10 min gas flow without a sample can be taken into account. The residual total moisture content may be further reduced by applying a second heating step to the calcium carbonate. In case said drying is carried out by more than one drying steps, the first step may be carried out by heating in a hot current of air, while the second and further drying steps are preferably carried out by an indirect heating in which the atmosphere in the corresponding vessel comprises a surface treatment agent. It is also common that the calcium carbonate is subjected to a beneficiation step (such as a flotation, bleaching or magnetic separation step) to remove impurities.

In one embodiment of the present invention, the ground calcium carbonate comprises a dry ground calcium carbonate. In another preferred embodiment, the ground calcium carbonate is a material being wet ground in a horizontal ball mill, and subsequently dried by using the well known process of spray drying.

The ground calcium carbonate may comprise, one or more, for example, two or three calcium carbonates. According to a preferred embodiment, the ground calcium carbonate comprises only one calcium carbonate, and preferably marble.

According to one embodiment of the present invention, the Pickering pigments, preferably the surface-reacted calcium carbonate particles are not coated with a surface treatment agent. According to a preferred embodiment, the Pickering pigments of the present invention are not surface treated with fatty acid esters such as glyceryl monostearate, PEG 7 glyceryl cocoate, glycol stearate or glycol distearate, lecithin, fractioned lecithin, hydrogenated lecithin, surfactants such as sodium cocoyl glycinate, castor oil derivatives such as 12-hydroxy stearic acid or hydrogenated castor oil, fatty alcohols such acetyl alcohol, ceto stearyl alcohol, stearyl alcohol or behenyl alcohol or saturated or unsaturated fatty acids such as myristic acid palmitic acid, stearic acid or oleic acid or salts thereof, mono- or di-substituted succinic anhydride containing compounds, mono- or di-substituted succinic acid containing compounds, mono- or di-substituted succinic acid salts containing compounds, unsaturated esters of phosphoric acid, salts of unsaturated phosphoric acid esters; mixtures thereof and reaction products thereof.

The Pickering pigments are present in the Pickering emulsion in an amount of 1 to 10 wt.-% based on the total weight of the Pickering emulsion. According to a preferred embodiment the Pickering emulsions comprise 2 to 10 wt.-% Pickering pigments, based on the total weight of the Pickering emulsion, preferably 4 to 10 wt.-% Pickering pigments and most preferably 6 to 10 wt.-% Pickering pigments.

The Pickering pigments are calcium carbonate particles selected from surface-reacted calcium carbonate (SRCC) or mixtures of ground calcium carbonate (GCC) and surface-reacted calcium carbonate (SRCC). According to a preferred embodiment of the present invention, the Pickering pigments only comprise surface-reacted calcium carbonate (SRCC).

Alternatively, the Pickering pigments comprise a mixture of ground calcium carbonate (GCC) and surface-reacted calcium carbonate (SRCC). In that case the ratio of GCC : SRCC is from 1:100 to 100:100 based on the dry weight of the GCC and the SRCC, preferably from 10:100 to 90:100, more preferably from 30:100 to 80:100, and most preferably from 50:100 to 70:100.

According to an exemplified embodiment of the present invention the Pickering pigments comprise surface-reacted calcium carbonate particles and preferably consist of surface-reacted calcium carbonate particles. Preferably, the surface-reacted calcium carbonate particles have a specific surface area of from 40 m²/g to 70 m²/g, measured using nitrogen and the BET method and a volume median particle size d₅₀ value from 5.0 µm to 8.0 µm.

The Pickering emulsions of the present invention can be oil-in-water emulsions or water-in-oil emulsions. A oil-in-water Pickering emulsion is an emulsion wherein the oil droplets are stabilized by the Pickering pigments in the water. A water-in-oil Pickering emulsion is an emulsion, wherein the water droplets are stabilized by the Pickering pigments in the oil. According to a preferred embodiment of the present invention, the Pickering emulsions of the present invention are oil-in-water emulsions.

According to an exemplified embodiment of the present invention the Pickering emulsion comprises (i) water, (ii) 10 to 50 wt.-% oil, based on the total weight of the Pickering emulsion, preferably selected from the group consisting of essential oils, sunflower oil, olive oil, palm oil, coconut oil, peanut oil, palm kernel oil, corn oil, hazelnut oil, sesame oil and mixtures thereof, more preferably selected from sunflower oil, olive oil, palm oil and/or coconut oil and most preferably sunflower oil and
(iii) 1 to 10 wt.-% of Pickering pigments, based on the total weight of the Pickering emulsion,
wherein the Pickering pigments are calcium carbonate particles selected from surface-reacted calcium carbonate (SRCC) or mixtures of ground calcium carbonate (GCC) and surface-reacted calcium carbonate (SRCC), preferably are surface-reacted calcium carbonate particles (SRCC) and
wherein the calcium carbonate particles have a volume median particle size d₅₀ value from 0.2 µm to 10 µm. According to a preferred embodiment the water in the Pickering emulsion is demineralized water.

According to another exemplified embodiment of the present invention, the Pickering emulsion comprises (i) water, preferably demineralized water, (ii) 10 to 50 wt.-% oil, based on the total weight of the Pickering emulsion, preferably sunflower oil and
(iii) 1 to 10 wt.-% of Pickering pigments, based on the total weight of the Pickering emulsion,
wherein the Pickering pigments are calcium carbonate particles selected from surface-reacted calcium carbonate (SRCC) or mixtures of ground calcium carbonate (GCC) and surface-reacted calcium carbonate (SRCC), preferably are surface-reacted calcium carbonate particles (SRCC) and
wherein the calcium carbonate particles have a volume median particle size d₅₀ value from 0.2 µm to 10 µm.

According to another exemplified embodiment of the present invention, the Pickering emulsion comprises (i) water, preferably demineralized water, (ii) 10 to 50 wt.-% oil, based on the total weight of the Pickering emulsion, preferably sunflower oil and
(iii) 1 to 10 wt.-% of Pickering pigments, based on the total weight of the Pickering emulsion, wherein the Pickering pigments are surface-reacted calcium carbonate particles (SRCC) and
wherein the calcium carbonate particles have a volume median particle size d₅₀ value from 1.5 µm to 9 µm and a specific surface area (BET) of from 10 to 200 m²/g, preferably from 20 to 180 m²/g, more preferably from 25 to 140 m²/g, for example from 40 to 70 m²/g as measured by the BET nitrogen method.

### Additional Embodiments

According to one embodiment of the present invention, the emulsions comprise further active ingredients, preferably selected from cosmetic active compounds, pharmaceutical active compounds, nutritional additives, flavoring agents and mixtures thereof. Such compounds are known to the skilled person and are commercially available. The skilled person can choose such compounds dependent on the used oil and the intended use of the Pickering emulsion.

“Cosmetic active compounds” in the meaning of the present invention are active ingredients in cosmetic products and have at least some positive or beneficial effects on the skin or the hair. Cosmetic active agents are known to the skilled person and are commercially available. The skilled person can choose such compounds dependent on the used oil and the intended use of the Pickering emulsion. Known cosmetic active agents are, for example, hyaluronic acid, vitamin E, vitamin C, kojic acid, AHAs, BHA, hydroquinone, vitamin A/retinoids, salicylic acid, benzoyl peroxide, azelaic acid or sulfur.

“Pharmaceutical active agents” in the meaning of the present invention are the ingredients in a pharmaceutical drug that are biologically active. Pharmaceutically active agents are known to the skilled person and are commercially available. The skilled person can choose such agents dependent on the used oil and the intended use of the Pickering emulsion. Known pharmaceutical active agents are, for example, vitamin A, vitamin D, vitamin C, polyphenols, caffeine, flavonoids, carotenoids, isoflavones or sterols.

“Nutritional additives” in the meaning of the present invention are additives that are added to foods or drinks for the purpose of restoring nutrients lost or degraded during production, fortifying or enriching certain foods or drinks in order to correct dietary deficiencies, or adding nutrients to food or drink substitutes. Nutritional additives are known to the skilled person and are commercially available. The skilled person can choose such additives dependent on the used oil and the intended use of the Pickering emulsion. Known nutritional additives are, for example, vitamin A, vitamin D, vitamin C, vitamin B, omega-3 oils, minerals like sodium, manganese or selenium.

“Flavoring agents” in the meaning of the present invention are ingredients that impart a flavor or a taste to a product, for example, food, drinks or medicine. Flavoring agents are known to the skilled person and are commercially available. The skilled person can choose such compounds dependent on the used oil and the intended use of the Pickering emulsion. The flavoring agents may be natural flavoring agents, nature-identical flavoring agents or synthetically flavoring agents. Known flavoring agents are, for example, manzanate, isoamyl acetate, benzaldehyde, cinnamaldehyde, ethyl proprionate, methyl anthranilate, limonene, ethyl dedacidienoate, allyl hexanoate, ethyl maltol or methyl salicylate.

The Pickering emulsions of the present invention have excellent stability against coalescence. By “stable against coalescence” it is meant that the emulsions, stored motionless between 4° C. and 20° C. do not exhibit an increase in the average droplet diameter of more than 10%.

A “droplet,” in the meaning of the present invention is an isolated portion of a first fluid that is completely surrounded by a second fluid. It is to be noted that a droplet is not necessarily spherical, but may assume other shapes as well, for example, depending on the external environment. The “average diameter” of a population of droplets is the arithmetic average of the diameters of the droplets. Those of ordinary skill in the art will be able to determine the average diameter of a population of droplets, for example, using laser light scattering or other known techniques. The diameter of a droplet, in a non-spherical droplet, is the mathematically-defined average diameter of the droplet, integrated across the entire surface.

According to a preferred embodiment of the present invention the Pickering emulsions are stable against coalescence for at least 15 days, more preferably for at least 20 days and most preferably for at least 30 days.

According to one embodiment of the present invention the Pickering emulsions of the present invention have an improved stability against coalescence, compared to identical Pickering emulsions that do not comprise the inventive Pickering pigments.

An “identical Pickering emulsion” in the meaning of the present invention refers to an Pickering emulsion that consists of the same ingredients in the same amounts than the inventive Pickering emulsions with the exception, that the emulsions do not comprise the inventive Pickering pigments but different Pickering pigments that are known in the prior art.

According to one embodiment of the present invention, the Pickering emulsions according to the present invention do not comprise an additional emulsifier for stabilizing the droplets in the Pickering emulsions apart from the Pickering pigments.

As already set out above, an “emulsifier” or “surface active agent” or “surfactant” or “surface treatment agent” is a substance that stabilizes an emulsion by increasing its kinetic stability. Emulsifiers are compounds that typically have an amphiphile molecular structure consisting of a polar (hydrophile) and a non-polar (lipophile) part of the molecule which are separated from each other in space. Conventional emulsifiers can be classified depending on their hydrophile part of the molecule into ionic (anionic, cationic and amphoteric) and non-ionic ones.

Emulsifiers are known to the skilled person and are commercially available. For example, an anionic emulsifier that is known to the skilled person is soap which is the conventional name for the water-soluble sodium or potassium salts of saturated and non-saturated higher fatty acids. A known cationic emulsifier is an quaternary ammonium compound. The hydrophilic part of the molecule of non-ionic emulsifiers often consists of glycerol, polyglycerol, sorbitanes, carbohydrates or polyoxyethylene glycols, respectively, and is most often connected to the lipophilic part of the molecule by means of ester and ether bonds. The latter consists typically of fatty alcohols, fatty acids or so-fatty acids.

By variation of the structure and the size of the polar and the non-polar part of the molecule, lipophilicity and hydrophilicity of emulsifiers can be modified to a large extent. The skilled person knows how to prepare and select emulsifiers dependent on the application.

As already set out above the Pickering emulsions according to the present invention do not comprise an additional emulsifier for stabilizing the droplets in the Pickering emulsions apart from the Pickering pigments. According to a preferred embodiment, the Pickering emulsions of the present invention do not comprise fatty acid esters such as glyceryl monostearate, PEG 7 glyceryl cocoate, glycol stearate or glycol distearate, lecithin, fractioned lecithin, hydrogenated lecithin, surfactants such as sodium cocoyl glycinate, castor oil derivatives such as 12-hydroxy stearic acid or hydrogenated castor oil, fatty alcohols such acetyl alcohol, ceto stearyl alcohol, stearyl alcohol or behenyl alcohol or saturated or unsaturated fatty acids such as myristic acid palmitic acid, stearic acid or oleic acid or salts thereof, mono- or di-substituted succinic anhydride containing compounds, mono- or di-substituted succinic acid containing compounds, mono- or di-substituted succinic acid salts containing compounds, unsaturated esters of phosphoric acid, salts of unsaturated phosphoric acid esters; mixtures thereof and reaction products thereof.

The inventors of the present invention surprisingly found out that the Pickering emulsions of the present invention have sufficient or improved properties.

First of all, the Pickering pigments in the Pickering emulsions of the present invention have a volume median particle size d₅₀ value from 0.2 µm to 10 µm and, therefore, do not comprise nanoparticles that have mainly primary diameters below 150 nm. This is advantageous since recent research has shown that such small nanoparticles may have disadvantages for the environment, for humans and animals since such small nanoparticles can enter organisms by ingestion or through the skin and can translocate within the body to various organs and tissues or within plants. Due to their reactivity with human, animals or plant cells they can show toxicological effects.

Furthermore, the inventors of the present invention have surprisingly found that the Pickering emulsion of the present invention do not need an additional emulsifier or surfactants, co-stabilizers or surface coatings on the surface of the Pickering pigments, for stabilizing the droplets in the Pickering emulsions apart from the Pickering pigments and, therefore, clean label emulsions can be produced. The addition of such emulsifiers, surfactants, co-stabilizers or surface coatings often is not desirable especially in Pickering emulsions that are used in agriculture or for humans and animals since such compounds can have side effect for humans or animals and may not be environmentally friendly.

The inventors furthermore found that the inventive Pickering emulsions have a white colour, even if a coloured, for example, a yellow oil is used.

### Composition Comprising the Pickering Emulsion

According to one embodiment of the present invention a composition comprising the inventive Pickering emulsion is provided, wherein the composition is a food composition, a cosmetic composition, a pharmaceutical composition or a nutritional formula.

The Pickering emulsion of the invention may be used in a food composition, for example a beverage as well as emulsions such as mayonnaise, low-fat spreads, vinaigrette, ice-cream, sauces and soups. For example Pickering emulsions may be used to introduce hydrophobic nutritional additives or flavouring agents into food or beverages. Such Pickering emulsions may encapsulate or protect sensitive and active food components from the environment, for example, against oxidation or may be used to control aroma and flavor release.

Cosmetic products can benefit from the range of textures that may be obtained with the Pickering emulsions of the present invention, as well as the possibility of incorporating cosmetic active agents such as fat soluble bioactive materials within the oil droplets.

In pharmaceutical compositions, Pickering emulsions may be used to protect sensitive pharmaceutically active agents, and to mask the unpleasant taste of some pharmaceutically active agents.

The composition comprising the Pickering emulsion of the invention may be a nutritional formula. The nutritional formula may be a complete nutritional formula which provides sufficient types and levels of macronutrients (protein, fats and carbohydrates) and micronutrients to be sufficient as a sole source of nutrition for the subject to which it is administered. The nutritional formula may also provide partial nutrition, to act as a supplement to the existing diet of the subject.

### Method for Preparing the Pickering Emulsion

According to the present invention a method of preparing the inventive Pickering emulsions is provided.

The method comprises the steps of:
A) providing water,
B) providing oil,
C) providing Pickering pigments, wherein the Pickering pigments are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from above 0.1 µm to 10 µm
D) combining the water of step A), the oil of step B) and the Pickering pigments of step C) in any order to obtain a mixture comprising 10 to 50 wt.-% oil, based on the total weight of the mixture and 1 to 10 wt.-% of Pickering pigments, based on the total weight of the mixture and
E) mixing the mixture obtained in step D) to prepare a Pickering emulsion.

It should be understood, that the method of the present invention may be carried out as a continuous process or as a batch process. Preferably, the inventive method is carried out as a batch process.

In the following, it is referred to further details of the present invention and especially to the foregoing steps of the inventive method for preparing the Pickering emulsions. The water, the oil and the Pickering pigments have already been defined above.

### Step D

In step D) of the manufacturing method according to the present invention, the water of step A), the oil of step B) and the Pickering pigments of step C) are combined in any order to obtain a mixture comprising 10 to 50 wt.-% oil, based on the total weight of the mixture and 1 to 10 wt.-% of Pickering pigments, based on the total weight of the mixture.

The contacting or combining of the water of step A), the oil of step B) and the Pickering pigments of step C) can be accomplished by any conventional means known to the skilled person.

According to one embodiment of the present invention, step D) comprises the steps of providing the water provided in step A) in a first step and then adding oil provided in step B) in a subsequent step. This mixture is combined with the Pickering pigments provided in step C) either by adding the liquid mixture to the Pickering pigments or by adding the Pickering pigments to the liquid mixture. According to another embodiment of the present invention, step D) comprises the steps of providing the oil provided in step B) in a first step and then adding water provided in step A) in a subsequent step. This mixture is combined with the Pickering pigments provided in step C) either by adding the liquid mixture to the Pickering pigments or by adding the Pickering pigments to the liquid mixture.

According to another embodiment of the present invention, step D) comprises the steps of providing the water provided in step A) in a first step and then adding the Pickering pigments provided in step C) in a subsequent step. This slurry is afterwards combined with the oil provided in step B) either by adding the oil to the slurry or by adding the slurry to the oil. According to another embodiment of the present invention, step D) comprises the steps of providing the Pickering pigments provided in step C) in a first step and then adding in a subsequent step the water provided in step A). This slurry is afterwards combined with the oil provided in step B) either by adding the oil to the slurry or by adding the slurry to the oil.

According to another embodiment of the present invention, step D) comprises the steps of providing the oil provided in step B) in a first step and then adding the Pickering pigments provided in step C) in a subsequent step. This slurry is afterwards combined with the water provided in step A) either by adding the water to the slurry or by adding the slurry to the water. According to another embodiment of the present invention, step D) comprises the steps of providing the Pickering pigments provided in step C) in a first step and then adding in a subsequent step the oil provided in step B). This slurry is afterwards combined with the water provided in step A) either by adding the water to the slurry or by adding the slurry to the water.

According to another embodiment of the present invention, step D) comprises the steps of combining the oil provided in step B) with a defined amount of the Pickering pigments provided in step C). The remaining amount of the Pickering pigments provided in step C) is combined with the water provided in step A). Afterwards both slurries are combined together in any order.

The Pickering pigments provided in step C) can be added to the water, or the oil or the mixture in one portion or may be added in several equal or unequal portions, i.e. in larger and smaller portions.

According to a preferred embodiment of the present invention step D) comprises the steps of providing the oil provided in step B) in a first step and then adding the Pickering pigments provided in step C) in a subsequent step. This slurry is afterwards combined with the water provided in step A) either by adding the water to the slurry or by adding the slurry to the water and preferably, by adding the water to the slurry.

### Step E

In step E) the mixture obtained in step D) is mixed to prepare a Pickering emulsion.

As already set out above a Pickering emulsion in the meaning of the present invention is an emulsion wherein the Pickering pigments accumulate in the oil/water boundary surface in the form of a layer whereby the joining of the dispersed phases is prevented.

The skilled person knows how to mix such mixtures in order to prepare a Pickering emulsion.

The mixing in step E) can be accomplished by any conventional means known to the skilled person that will result in a Pickering emulsion. The skilled person will adapt the mixing conditions such as the mixing speed, dividing, and temperature according to his process equipment.

For example, mixing may be performed by use of a disperser/homogenizer. Equipment that may be used in the inventive process is commercially available, for example, from IKA, Germany, under the trade name ULTRA-TURRAX, for example, ULTRA-TURRAX T10 basic, from GEA under the trade name Ariete Homogenizer 5400, mixers from Silverson, for example, the Ultramix or sonication devices from hielscher, for example the UP200 ST.

According to another embodiment of the present invention, step E) is carried out for at least 1 second, preferably for at least 1 minute (e.g. 10 min, 30 min or 60 min). According to a preferred embodiment step (c) is carried out for a period of time ranging from 1 second to 60 min, preferably for a period of time ranging from 15 min to 45 min. For example, mixing step (d) is carried out for 30 min ± 5 min.

According to another embodiment of the present invention, step E) is carried out at room temperature, preferably at temperatures between 15 to 25° C. However, step E) can also be carried out at lower or higher temperatures, for example in a temperature range of 4° C. to 95° C., preferably in a temperature range of 10° C. to 70° C., most preferably in a temperature range of 15° C. to 40° C.

According to one embodiment of the present invention the Pickering pigments provided in step C) and preferably of the surface-reacted calcium carbonate particles are not coated with a surface treatment agent.

According to one embodiment of the present invention, the Pickering pigments, preferably the surface-reacted calcium carbonate particles are not coated with a surface treatment agent. According to a preferred embodiment the Pickering pigments of the present invention are not surface treated with fatty acid esters such as glyceryl monostearate, PEG 7 glyceryl cocoate, glycol stearate or glycol distearate, lecithin, fractioned lecithin, hydrogenated lecithin, surfactants such as sodium cocoyl glycinate, castor oil derivatives such as 12-hydroxy stearic acid or hydrogenated castor oil, fatty alcohols such acetyl alcohol, ceto stearyl alcohol, stearyl alcohol or behenyl alcohol or saturated or unsaturated fatty acids such as myristic acid palmitic acid, stearic acid or oleic acid or salts thereof, mono- or di-substituted succinic anhydride containing compounds, mono- or di-substituted succinic acid containing compounds, mono- or di-substituted succinic acid salts containing compounds, unsaturated esters of phosphoric acid, salts of unsaturated phosphoric acid esters; mixtures thereof and reaction products thereof.

The inventors surprisingly found that by the above method it is possible to prepare the inventive Pickering emulsions. The above method is a cheap and especially easy to handle method and the inventive Pickering emulsion can be easily and quickly produced by the inventive method.

### Use of Calcium Carbonate Particles

According to one aspect of the present invention calcium carbonate particles are used as Pickering pigments for stabilizing Pickering emulsions comprising water and 10 to 50 wt.-% oil, based on the total weight of the Pickering emulsion, wherein the calcium carbonate particles are selected from surface-reacted calcium carbonate (SRCC) or mixtures of ground calcium carbonate (GCC) and surface-reacted calcium carbonate (SRCC) and have a volume median particle size d₅₀ value from 0.2 µm to 10 µm.

The water, the oil and the Pickering pigments as well as the Pickering emulsions have already been defined above.

The inventors of the present invention surprisingly found out that the above described Pickering pigments can be used in Pickering emulsions.

The Pickering pigments that can be used in the Pickering emulsions of the present invention have a volume median particle size d₅₀ value from 0.2 µm to 10 µm and, therefore, do not comprise nanoparticles that have primary diameters below 150 nm. This is advantageous since recent research has shown that such small nanoparticles may have disadvantages for the environment, for humans and animals due to the fact that small nanoparticles can enter organisms during ingestion or through the skin and can translocate within the body to various organs and tissues or within plants. Due to their reactivity with human, animals or plants cells they can show toxicological effects.

Furthermore, the inventors of the present invention have surprisingly found that when the above mentioned Pickering pigments are used, the Pickering emulsion of the present invention do not need additional emulsifiers or surfactants, co-stabilizers or surface coatings on the surface of the Pickering pigments, for stabilizing the droplets in the Pickering emulsions apart from the Pickering pigments and, therefore, clean label emulsions can be produced. The addition of such emulsifiers, surfactants, co-stabilizers or surface coatings often is not desirable especially in Pickering emulsions that are used in agriculture or for humans and animals since such compounds can have side effect for humans or animals and may not be environmentally friendly.

The inventors furthermore found that the inventive Pickering pigments have a white colour, even if a coloured, for example, a yellow oil is used.

The scope and interest of the invention will be better understood based on the following examples which are intended to illustrate certain embodiments of the present invention and are non-limitative.

### FIGURES

FIG. 1: Microscope image of Pickering emulsion 2

FIG. 2: Microscope image of Pickering emulsion 3

FIG. 3: Droplet size dependent on storage time of Pickering emulsion 1

### EXPERIMENTS

### 1. Measurement Methods

In the following, measurement methods implemented in the examples are described.

### BET Specific Surface Area (SSA) of a Material

The BET specific surface area was measured via the BET process according to ISO 9277:2010 using nitrogen, following conditioning of the sample by heating at 250° C. for a period of 30 minutes. Prior to such measurements, the sample was filtered, rinsed and dried at 110° C. in an oven for at least 12 hours.

### Particle Size Distribution (% Particles With a Diameter < X), D₅₀ Value (Median Grain Diameter) and d₉₈ Value of a Particulate Material

Volume median grain diameter d₅₀(vol) was evaluated using a Malvern Mastersizer 2000 Laser Diffraction System or a Malvern Mastersizer 3000 Laser Diffraction System. The d₅₀(vol) or d₉₈(vol) value, measured using a Malvern Mastersizer 2000 Laser Diffraction System or Malvern Mastersizer 3000 Laser Diffraction System, indicates a diameter value such that 50 % or 98 % by volume, respectively, of the particles have a diameter of less than this value. The raw data obtained by the measurement are analysed using the Mie theory, with a particle refractive index of 1.57 and an absorption index of 0.005.

The processes and instruments are known to the skilled person and are commonly used to determine grain size of fillers and pigments.

The weight determined or based median grain diameter d₅₀(wt) was measured by the sedimentation method, which is an analysis of sedimentation behaviour in a gravimetric field. The measurement was made with a Sedigraph™ 5120 of Micromeritics Instrument Corporation, USA. The method and the instrument are known to the skilled person and are commonly used to determine particle size distributions of fillers and pigments. The measurement was carried out in an aqueous solution of 0.1 wt.-% Na₄P₂O₇. The samples were dispersed using a high speed stirrer and supersonicated.

### Porosity / Pore Volume

The porosity or pore volume is measured using a Micromeritics Autopore IV 9500 mercury porosimeter having a maximum applied pressure of mercury 414 MPa (60 000 psi), equivalent to a Laplace throat diameter of 0.004 µm (~ nm). The equilibration time used at each pressure step is 20 seconds. The sample material is sealed in a 5 ml chamber powder penetrometer for analysis. The data are corrected for mercury compression, penetrometer expansion and sample material compression using the software Pore-Comp (Gane, P.A.C., Kettle, J.P., Matthews, G.P. and Ridgway, C.J., “Void Space Structure of Compressible Polymer Spheres and Consolidated Calcium Carbonate Paper-Coating Formulations”, Industrial and Engineering Chemistry Research, 35(5), 1996, pp 1753-1764.).

### Type of Pickering Emulsion

The drop method was used to determine the type of emulsion. One drop of the emulsion was placed in water and one in oil. In the medium of the continuous phase, the emulsions are dispersible, in the medium of the disperse phase the drops settle on the vessel wall. This means O/W emulsions can be dispersed in water but not in oil and W/O emulsions can only be dispersed in oil.

### Droplet Size (Optical and Light Scattering)

The droplet size is determined by microscopic analysis. Under the microscope (Olympus BX51, Olympus Europa SE& Co KG, Germany), images are taken at two different points on each sample (Olympus SC50, Olympus Europa SE& Co KG, Germany) and ten droplets are measured using cellSens software. From these 20 measured values, the mean value and the standard deviation are determined.

In addition to the optical evaluation, the droplet size was also determined by light scattering (Mastersizer 3000, Malvern Panalytical GmbH, Germany). For this purpose 2 mL of the emulsion was diluted with water and added to the wet dispersion module for measurement. The evaluation was carried out using Mie theory for round particles and a refractive index of 1.53, which lies between the value for sunflower oil and that for calcium carbonate. Thus the multiple refraction at a Pickering drop (particle-drop particle) is taken into account.

The measured values determined by light scattering do not differ from the optical evaluation.

### 2. Material and Equipment

### Materials

Water: Demineralized water
Oil: Sunflower oil, available from M-classic
Pickering pigment: surface-reacted calcium carbonate (SRCC) (d₅₀(vol) = 6.6 µm, d₉₈(vol) = 13.7 µm, SSA = 59.9 m²/g). The intra-particle intruded specific pore volume is 0.939 cm³/g (for the pore diameter range of 0.004 to 0.51 µm).

SRCC was obtained by preparing 350 litres of an aqueous suspension of ground calcium carbonate in a mixing vessel by adjusting the solids content of a ground limestone calcium carbonate from Omya SAS, Orgon having a weight based median grain diameter d₅₀(wt) of 1.3 µm, as determined by sedimentation, such that a solids content of 10 wt.-%, based on the total weight of the aqueous suspension, is obtained.

Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.
Ground calcium carbonate I (GCC I) (d₅₀(vol) = 1.0 µm, SSA = 3.7 m²/g).
Ground calcium carbonate II (GCC II) (d₅₀(vol) = 8.1 µm, SSA = 2.1 m²/g).
Precipitated calcium carbonate (PCC) (d₅₀(vol) = 7.65 µm, SSA = 3.3 m²/g).

### Preparation of the Pickering Emulsions 1 to 3

The oil is added to a glass beaker. Afterwards the Pickering pigment (surface reacted calcium carbonate as mentioned above) is added to the oil and dispersed with a high shear mixer (Polytron PT3100D, Kinematica AG, Switzerland) for 1 minute at 5000 rpm. Subsequently water is added slowly to the slurry over 1 minute and the mixture is homogenized for 4 minutes at 15000 rpm with a high shear mixer (Polytron PT3100D, Kinematica AG, Switzerland).

The used amounts are given in table 1 below

TABLE 1

| | Oil content [g] | Pigment content [g] | Water content [g] |
|---|---|---|---|
| Pickering emulsion 1 | 75 | 12 | 63 |
| Pickering emulsion 2 | 15 | 1.5 | 133.5 |
| Pickering emulsion 3 | 45 | 4.5 | 100.5 |

### Preparation of the Emulsions 4 and 5

The emulsions 4 and 5 were prepared by the method according to the Pickering emulsions 1 to 3. As pigment ground calcium carbonate I (GCC I) has been used.

TABLE 2

| | Oil content [g] | Pigment content [g] | Water content [g] |
|---|---|---|---|
| Emulsion 4 | 75 | 10.5 | 64.5 |
| Emulsion 5 | 75 | 15 | 60 |

### Preparation of the Pickering Emulsions/Emulsions 6 to 17

The oil is added to a glass beaker. Afterwards the Pickering pigment (SRCC) (emulsions 10, 11, 16, 17) or the ground calcium carbonate II (GCC II) (emulsions 8, 9, 14, 15) or the precipitated calcium carbonate (emulsions 6, 7, 12, 13) is added to the oil and dispersed with a high shear mixer (Ultra Turrax T25, IKA®-Werke GmbH & CO. KG, Germany) for 30 seconds at 6500 rpm. Subsequently water is added slowly to the slurry over 30 seconds and the mixture is homogenized for 2 minutes at 17500 rpm with a high shear mixer (Ultra Turrax T25, IKA®-Werke GmbH & CO. KG, Germany).

| | Oil content [g] | Pigment content [g] | Water content [g] |
|---|---|---|---|
| Emulsion 6 | 22.5 | 3.75 | 48.75 |
| Emulsion 7 | 22.5 | 7.5 | 45 |
| Emulsion 8 | 22.5 | 3.75 | 48.75 |
| Emulsion 9 | 22.5 | 7.5 | 45 |
| Pickering emulsion 10 | 22.5 | 3.75 | 48.75 |
| Pickering emulsion 11 | 22.5 | 7.5 | 45 |
| Emulsion 12 | 37.5 | 3.75 | 33.75 |
| Emulsion 13 | 37.5 | 7.5 | 30 |
| Emulsion 14 | 37.5 | 3.75 | 33.75 |
| Emulsion 15 | 37.5 | 7.5 | 30 |
| Pickering emulsion 16 | 37.5 | 3.75 | 33.75 |
| Pickering emulsion 17 | 37.5 | 7.5 | 30 |

In emulsions 6, 8, 9, 12, 13, 14 and 15 the oil and water phase do not form an emulsion but rather each phase is present separately in the mixture.

### 3. Example Data

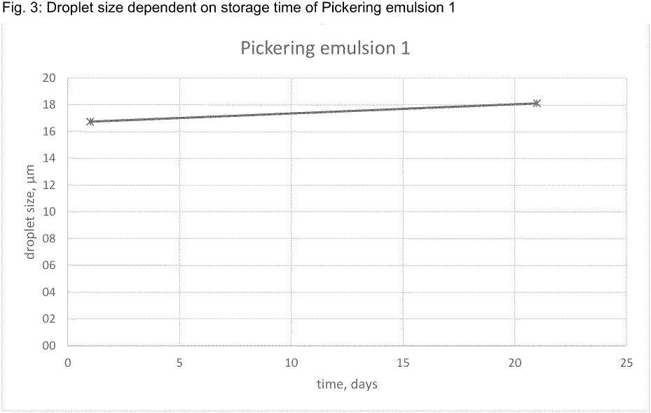

The droplet size of Pickering emulsion 1 has been measured by light scattering on the day of the preparation and after storing for 21 days at room temperature. As can be seen from FIG. 3 the droplet size merely changes from 17 µm to 18 µm and, therefore, is stable against coalescence.

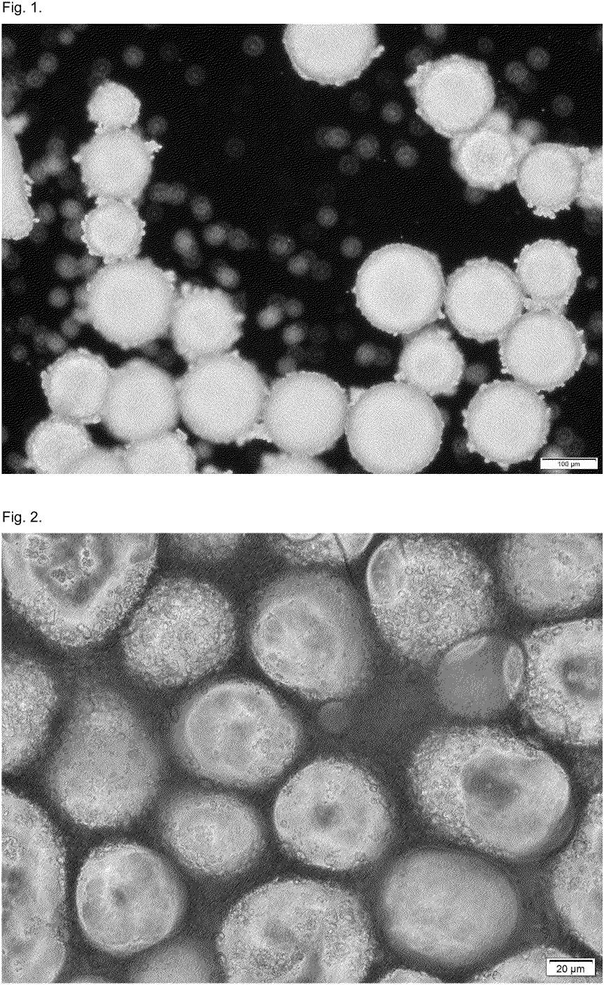

The droplet size of Pickering emulsions 2 and 3 has been determined by microscopic analysis on the day of the preparation at room temperature. As can be seen from FIGS. 1 and 2 the droplet size is very homogeneous and varies from about 100 µm to 150 µm in Pickering emulsion 2 and from about 30 to 70 µm in Pickering emulsion 3.

The droplet size of emulsions 4 and 5 has been determined by microscopic analysis on the day of the preparation at room temperature. The droplet size is not very homogeneous and varies from about 30 µm to 150 µm in Emulsion 4 and from about 20 to 80 µm in Emulsion 5.

Both emulsions prepared merely with GCC (emulsions 4 and 5) were instable after 14 days with a separated oil phase. Therefore no droplet size measurement was possible after that storage time.

The droplet size of Pickering emulsions/emulsions 6 to 17 has been measured by light scattering after storing for 21 days at room temperature (if an emulsion has been formed). Emulsions 6, 8, 9, 12, 13, 14 and 15 do not form an emulsion but rather each phase (the oil phase and water phase) is present separately in the mixture. The droplet size of emulsion 7 is 305 µm, of emulsion 10 63 µm, of emulsion 11 25 µm, of emulsion 16 145 µm and of emulsion 17 54 µm.

The above experiments show that it is possible to prepare stable Pickering emulsions with Pickering pigments that have a volume median particle size d₅₀ value from 0.2 µm to 10 µm and, therefore, do not comprise nanoparticles that have mainly primary diameters below 150 nm. Furthermore, as can be seen from the above experiments, these Pickering emulsion do not need an additional emulsifier or surfactants, co-stabilizers or surface coatings on the surface of the Pickering pigments, for stabilizing the droplets in the Pickering emulsions apart from the Pickering pigments. Additionally, the inventive Pickering emulsions have a white colour. However, it is not possible to prepare such Pickering emulsions merely with ground calcium carbonate (GCC) or merely with precipitated calcium carbonate (PCC). Only with the inventive Pickering pigments it is possible to prepare stable Pickering emulsions that comprise water; 10 to 50 wt.-% oil, based on the total weight of the Pickering emulsion and 1 to 10 wt.-% of Pickering pigments, based on the total weight of the Pickering emulsion and are stable within the claimed range.

1. Pickering emulsion comprising
(i) water;
(ii) 10 to 50 wt.-% oil, based on the total weight of the Pickering emulsion and
(iii) 1 to 10 wt.-% of Pickering pigments, based on the total weight of the Pickering emulsion,
wherein the Pickering pigments are calcium carbonate particles selected from surface-reacted calcium carbonate (SRCC) or mixtures of ground calcium carbonate (GCC) and surface-reacted calcium carbonate (SRCC) and
wherein the calcium carbonate particles have a volume median particle size d₅₀ value from 0.2 µm to 10 µm.

2. The Pickering emulsion according to claim 1, wherein
the ground calcium carbonate is selected from the group consisting of marble, limestone, and/or chalk and preferably is marble and/or
the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source.

3. The Pickering emulsion according to claim 1, wherein the ground calcium carbonate has
a) a volume median particle size d₅₀ value from 0.3 µm to 5.0 µm, preferably from 0.6 µm to 3 µm and most preferably from above 1.0 µm to 1.7 µm, and/or
b) a top cut (d₉₈(vol)) of ≤20 µm, preferably ≤ 15 µm, more preferably ≤ 10 µm and most preferably ≤ 7 µm, and/or
c) a specific surface area (BET) of from 0.5 to 50 m²/g, preferably from 0.5 to 35 m²/g, more preferably from 0.5 to 25 m²/g, and most preferably from 0.6 to 17 m²/g, as measured by the BET nitrogen method.

4. The Pickering emulsion according to claim 1, wherein the surface-reacted calcium carbonate has
a) a volume median particle size d₅₀ value from 1.5 µm to 9.0 µm, preferably from 2.5 µm to 7.5 µm and most preferably from 3.3 µm to 6.6 µm, and/or
b) a top cut (d₉₈(vol)) of ≤ 20 µm, preferably ≤ 15 µm, more preferably ≤ 10 µm and most preferably ≤ 7 µm, and/or
c) a specific surface area (BET) of from 10 to 200 m²/g, preferably from 20 to 180 m²/g, more preferably from 25 to 140 m²/g, and most preferably from 48 to 110 m²/g, as measured by the BET nitrogen method and/or
d) an intra-particle intruded specific pore volume in the range from 0.1 to 2.3 cm³/g, more preferably from 0.2 to 2.0 cm³/g, especially preferably from 0.4 to 1.5 cm³/g, and most preferably from 0.6 to 1.1 cm³/g, calculated from mercury porosimetry measurement.

5. The Pickering emulsions according to claim 1, wherein the emulsions comprise 10 to 40 wt.-% oil, based on the total weight of the Pickering emulsion, preferably 10 to 30 wt.-% oil and most preferably 10 to 20 wt.-% oil.

6. The Pickering emulsions according to claim 1, wherein the oil
is selected from the group consisting of mineral oils, vegetable oils, animal fats, essential oils and mixtures thereof, preferably selected from the group consisting of essential oils, sunflower oil, olive oil, palm oil, coconut oil, peanut oil, palm kernel oil, corn oil, hazelnut oil, sesame oil and mixtures thereof, preferably is selected from sunflower oil, olive oil, palm oil and/or coconut oil and most preferably is sunflower oil and/or
is a refined oil having an acid value below 0.6, preferably below 0.5 and most preferably below 0.3 or an unrefined oil having an acid value below 4.0, preferably below 3.0 and most preferably below 2.0.

7. The Pickering emulsions according to claim 1, wherein the emulsions comprise 2 to 10 wt.-% Pickering pigments, based on the total weight of the Pickering emulsion, preferably 4 to 10 wt.-% Pickering pigments and most preferably 6 to 10 wt.-% Pickering pigments.

8. The Pickering emulsions according to claim 1, wherein the emulsions comprise further active ingredients, preferably selected from cosmetic active compounds, pharmaceutical active compounds, nutritional additives, flavoring agents and mixtures thereof.

9. The Pickering emulsions according to claim 1, wherein the emulsions are stable against coalescence for at least 15 days, more preferably for at least 20 days and most preferably for at least 30 days.

10. The Pickering emulsions according to claim 1, wherein the emulsion does not comprise an additional emulsifier for stabilizing the droplets in the Pickering emulsions apart from the Pickering pigments.

11. Composition comprising the Pickering emulsion of claim 1, wherein the composition is a food composition, a cosmetic composition, a pharmaceutical composition or a nutritional formula.

12. Method of preparing a Pickering emulsion, the method comprising the steps of:
A) providing water,
B) providing oil,
C) providing Pickering pigments, wherein the Pickering pigments are calcium carbonate particles selected from surface-reacted calcium carbonate (SRCC) or mixtures of ground calcium carbonate (GCC) and surface-reacted calcium carbonate (SRCC) and wherein the calcium carbonate particles have a volume median particle size d₅₀ value from above 0.1 µm to 10 µm
D) combining the water of step A), the oil of step B) and the Pickering pigments of step C) in any order to obtain a mixture comprising 10 to 50 wt.-% oil, based on the total weight of the mixture and 1 to 10 wt.-% of Pickering pigments, based on the total weight of the mixture and
E) mixing the mixture obtained in step D) to prepare a Pickering emulsion.

13. Method according to claim 12, wherein the surface of the Pickering pigments provided in step C) and preferably of the surface-reacted calcium carbonate particles is not coated with a surface treatment agent.

14. A method for stabilizing a Pickering emulsion, comprising adding water, 10 to 50 wt.-% oil based on the total weight of the Pickering emulsion, and calcium carbonate particles as Pickering pigments, wherein the calcium carbonate particles are selected from surface-reacted calcium carbonate (SRCC) or mixtures of ground calcium carbonate (GCC) and surface-reacted calcium carbonate (SRCC) and have a volume median particle size d₅₀ value from 0.2 µm to 10 µm.
